(19) United States
(12) Patent Application Publication
(10) Pub. No.: US 2022/0330788 A1
(43) Pub. Date: Oct. 20, 2022

(54) ON-BODY ANTENNA FOR WIRELESS COMMUNICATION WITH MEDICAL IMPLANT

(71) Applicant: OSLO UNIVERSITETSSYKEHUS HF, Oslo (NO)

(72) Inventors: Ali KHALEGHI, Trondheim (NO); Ilangko BALASINGHAM, Oslo (NO)

(21) Appl. No.: 17/641,158

(22) PCT Filed: Sep. 10, 2020
(86) PCT No.: PCT/EP2020/075383

§ 371 Date: Mar. 8, 2022

(30) Foreign Application Priority Data
Sep. 11, 2019 (GB) 1913086.3

(51) Int. Cl. A61B 1/00; A61B 1/04; A61B 5/0507; A61B 5/00; H01Q 1/27; H04W 4/80; H04B 1/3827

(52) U.S. Cl. CPC A61B 1/00016; A61B 1/041; A61B 5/0507; A61B 5/0031; H01Q 1/273; H04W 4/80; H04B 1/385

(57) ABSTRACT

A system is provided for wireless transmission of data and/or power using an on-body antenna apparatus (40) and an implant device inside the body. The system comprises the implant device and the on-body antenna apparatus (40) as well as an antenna control system. The implant device, is for use within the body and comprises an implant antenna (16) arranged to receive wirelessly transmitted power and/or to wirelessly transmit data. The on-body antenna apparatus (40) is arranged to transmit power and/or data acting as a radiative antenna, wherein the on-body antenna apparatus (40) comprises a pair of patch antennas (42) arranged to be placed on the surface of the body (44) spaced apart from one another to form an antenna circuit that is coupled by the body tissue around and between the patch antennas (42). The antenna control system is for providing power to the on-body antenna apparatus (40) and/or for handling communications between the on-body antenna apparatus (40) and the implant antenna (16), wherein the antenna control system is arranged to drive the on-body antenna apparatus (40).

The present invention relates to a system including an on-body antenna and a medical implant arranged for wireless communication of power and/or data between the on-body antenna and the implant, as well as to related methods.

Medical implants are used to gather information about the body and to interact with the body in various contexts. For example, capsule endoscopes are used to gather images within the digestive systems as well as to obtain samples or deliver drugs, neural prosthetic systems link the brain with external devices and exchange electrical signals with the brain, cardiac pacemakers or cardiac leadless pacemakers are used to synchronize the heart beats and various other devices have been proposed that rely on being held within the body or passed through the body. For all of these medical implant devices it provides advantages if there can be wireless communication with other devices outside or inside the body, and/or wireless power transfer, such as to provide power to the implant. Communication of data may be for wireless control or programming of the implant, for transmission of data from sensors such as cameras, temperature sensors, blood monitoring sensors and the like, and other things.

For a fully wireless connection from outside the body to the location of the implant device challenges can be generated both by the need for a minimum data transfer rate and by the depth of the body tissue.

Implant devices with wireless communication capabilities and also wireless power transfer have been proposed in the prior art. WO 2018/011235 describes the present inventors' proposals for an implant device using resonant antenna at the implant with a switch on the antenna to allow for backscatter communication, along with an on-body antenna apparatus comprising one or more loop antennas. The on-body antenna can also be used for wireless power transfer to the implant. With such systems it is valuable to ensure that the data transfer rate can be sufficiently high, as well as allowing for adequate range for communication and power transfer between the on-body antenna and the implant antenna.

Viewed from a first aspect, the invention provides a system for wireless transmission of data and/or power using an on-body antenna apparatus and an implant device inside the body, the system comprising:

the implant device, which is for use within the body and comprises an implant antenna arranged to receive wirelessly transmitted power and/or to wirelessly transmit data;

the on-body antenna apparatus, which is arranged to transmit power and/or data acting as a radiative antenna, wherein the on-body antenna apparatus comprises a pair of patch antennas arranged to be placed on the surface of the body spaced apart from one another and to form an antenna circuit that is coupled by the body tissue around and between the patch antennas; and

an antenna control system for providing power to the on-body antenna apparatus and/or for handling communications between the on-body antenna apparatus and the implant antenna, wherein the antenna control system is arranged to drive the on-body antenna apparatus.

The use of patch antennas to form an antenna circuit that is coupled by the body tissue increases the performance of the on-body antenna apparatus. By using the patch structures spaced apart by a suitable distance there can be coupling of a high frequency signal inside the tissues, making use of a signal path established between the patches and through the surrounding tissues, such as the tissues beneath the skin where the patches are placed, in use. This may extend the antenna range inside the tissues and hence extend the range for communication and/or power transfer with the implant. The antenna control system may be arranged to receive, via the on-body antenna apparatus, data transmitted from the implant device.

It will thus be understood that a significant advantageous feature of the proposed on-body antenna apparatus is to make use of the conductivity of the biological medium as a part of the antenna circuit. The on-body antenna of example embodiments couples RF energy to the conductive human body medium using a gap capacitance, and hence with a gap between the patch antennas and the surface of the body. This gap may be in the range 0.5 mm to 5 mm. The gap may be provided through the use of layer of non-conductive material between the patch antennas and the body surface, such as a separate layer or a coating on the patch antennas. The non-conductive material may be a suitable biocompatible material, for example a polymer material.

The efficient coupling that results from the proposed arrangement has several benefits/applications in different fields of RF technology for medical usage. Examples include:

1—Wireless power transfer (WPT) to medical implants.

2—Wireless communication with implants.

3—RFID and backscatter communications for implants.

4—Microwave medical imaging and tomography (narrowband and ultra-wideband).

5—Medical radar imaging techniques (narrowband and wideband).

6—Ultra-wideband communications.

7—Microwave hyperthermia.

8—MRI RF antennas

The above applications can be used in different medical fields, examples include but not limited to heart implant communication and WPT to the heart implants, heart imaging using Doppler and UWB radar, neck and breast microwave hyperthermia, wireless capsule endoscopy (WCE), neuro implants, struck detection based on tomographic approaches and microwave near-field imaging.

The on-body antenna may be a broadband antenna that has an antenna bandwidth covering Medical Implant Communication Service (MICS) bands and also being appropriate for wideband/ultrawideband applications. The Industrial, Scientific and Medical (ISM) frequency band, and Medical Implant Communication Service (MICS) at 400, 600, 800 and 1400 MHz are used for wireless communication with medical implants and the external communication system may hence use one of these frequencies.

The system may be arranged to operate at radio frequencies. For example, the system may be arranged to operate in the range 50 MHz to 1400 MHz, optionally in the range 100 MHz to 800 MHz. The system may be configured for transmissions in a MICS band such as for transmissions using frequencies in a MICS band, such as with frequencies in one or more bands known for medical implant use in the range 400 MHz-460 MHz, such as one or more of 401-406 MHz, 413-419 MHz, 426-432 MHz, 438-444 MHz, and 451-457 MHz.

As a result of the use of body tissue within an antenna circuit the size of the patch antennas can be small relative to the wavelength of the wirelessly transmitted power and/or data. In relation to the wavelength in free space, the maximum dimension of the patch antennas, e.g. the largest length, width or diameter, may be less than 20% of the wavelength, optionally less than 10% of the wavelength. Thus, for transmissions using frequencies in the ranges discussed above the size of the on-body antenna patches may use maximum dimensions in the range 5 mm to 100 mm, optionally 10 mm to 50 mm, with the particular size and configuration of the patches being determined based on the required frequencies of operation and the bandwidth that is needed.

The on-body antenna includes at least two patch antennas that are configured to be used spaced apart from one another on the body. The patch antennas may be held in a support structure that is arranged to keep them at a required spacing whilst mounted to the body surface and/or whilst not mounted to the body surface. The spacing between the antennas may be in the range 5 mm to 60 mm, optionally 10 mm to 40 mm, and the size of the spacing may be selected based on the intended frequency of operation as well as the size of the patches.

As the on-body antenna may make use of a natural gap to the underlying body surface, i.e. the gap of 0.5 mm to 5 mm discussed above, then advantageously the system does not need to include a conductive gel or the like between the on-body antenna apparatus and the body. It can communicate with the implant device without the use of such coupling gels, which has advantages for ease of use.

The patch antennas may include a first flat conductive patch, such as a rectangular patch, for placement on the body surface. They may further include an additional flat conductive patch extending away from the first flat conductive patch, for example at a right angle. The additional flat conductive patch may be a rectangular patch. Thus, the patch antennas may have an L-shape in cross section. The connections for feed points of the antennas may be on the additional flat conductive patch, such as on the L-shape at the furthest point from the first flat conductive patch, i.e. the furthest point on the antenna structure from the body surface. With this form the pair of patch antennas should be placed on the body surface with the additional flat conductive patches facing each other, and the first flat conductive patches each extending away additional flat conductive patches along the body surface in a direction away from the location of the other antenna.

The size of the patch antenna, which may be the side of the first flat conductive patch discussed above, may be in the range 5 mm to 100 mm, optionally 10 mm to 60 mm for example a rectangle with length and width in that range. In some particular examples, the dimensions of each patch antenna in the pair of patch antennas may be: 10 mm by 10 mm, such as for use with a frequency range of 450-1000 MHz, 20 mm by 10 mm, such as for use with a frequency range of 600-900 MHz, or 22.5 mm by 50 mm, such as for use with a frequency range of 250-350 MHz. In each case the two patches may be spaced apart with a gap in the range discussed above, such as about 40 mm.

The on-body antenna apparatus may comprise more than two patches. For example, there may be multiple pairs of patches. In one possible arrangement the on-body antenna apparatus includes multiple pairs of patches with different polarisation angles, for example by having multiple pairs spaced apart around the circumference of a circle, with each pair including a first patch at one point on the circumference and a second patch on another point on the circumference diametrically opposite the first patch. In another possible arrangement, the on-body antenna apparatus comprises a multiple pairs of patches forming an array, for example where the patches form a square grid.

Where the on-body antenna apparatus comprises a multiple pairs of patches forming an array there may be ten or more pairs of patches, with each pair being spaced apart from adjacent pairs. This allows for the on-body antenna apparatus to cover a larger area of the body than if only a single pair of patches was used. The antenna control system may be arranged to select one pair of patches within the array of pairs to use in relation to data or power communications with the implant device, for example in order to use the pair with the best connection to the implant. The antenna control system may be arranged to conduct a searching operation by switching between the pairs to find the best connection. The antenna control system may be arranged to feed respective pairs of patch antennas using a dual polarization scheme, and by switching between the pairs it becomes possible to add polarization switched diversity.

In some examples the implant device is capable of wireless transmission of data, optionally also with the ability to receive wirelessly transmitted power and/or wirelessly transmitted data from the on-body antenna. The implant device may include a data source for providing the data to be transmitted to the on-body antenna. The wireless transmission of data by the implant may be done using a backscatter communication technique, optionally via a system involving switching on the antenna as in WO 2018/011235, which may be implemented using a resonant antenna arrangement as in WO 2018/011235 or using a non-self-resonant implant antenna as described in further detail below.

Thus, in some examples the implant device is arranged to control the backscattering properties of the implant antenna in order to thereby transmit data to the on-body antenna apparatus; and the backscattering properties of the implant antenna are controlled by one or more electrical switch(es) that change the load and/or geometry of the implant antenna.

The implant antenna may have a configuration for backscatter communications that comprises a meandering path extending from a start of the meander at a capacitive coupling fed by a coupling patch to an end of the meander connected to a ground plane. The implant antenna may thus be a resonant antenna.

In other examples, the implant antenna is a non-self-resonant implant antenna configured for backscatter communications with the on-body antenna apparatus. The non-self-resonant implant antenna may comprise at least two electrodes including two conductive patches that are arranged to be spaced apart when the implant device is in use; wherein the implant device is arranged to control the backscattering properties of the non-self-resonant implant antenna in order to thereby transmit data to the on-body antenna apparatus. The implant device may be arranged such that the backscattering properties of the non-self-resonant antenna are controlled by one or more electrical switch(es) including an electrical switch that is arranged to change the impedance of the non-self-resonant implant antenna by switching between coupling the at least two electrodes via body tissue and coupling the at least two electrodes via a conductive pathway.

The non-self-resonant implant antenna can be controlled with very low power required at the implant device and with the possibility of a high change in implant antenna impedance, which allows for effective backscattering communication with the on-body antenna apparatus. The use of a non-self-resonant implant antenna allows for a large radar cross section (RCS) and hence for efficient backscatter performance. The switch changes the impedance of the non-self-resonant implant antenna by changing the degree to which the surrounding body tissue impacts on the effective impedance of the implant antenna. Thus, the switch can be open in order that the body tissue is a primary part of the coupling within an antenna circuit of the implant antenna, or the switch can be closed so that the two patches are coupled by the conductive pathway and the body tissue has minimal impact since it is only within the implant antenna circuit in “parallel” with the conductive pathway. It will be appreciated that by this approach, and the use of two patch antennas with a spacing between, it becomes possible to create a significant change in backscattering properties with minimal use of power at the implant device.

In this context self-resonance means that an antenna structure and geometry is designed in a way to provide resonance (as used in common antenna methodology). With non-self-resonance the antenna itself has no resonance but the impedance of the medium around the antenna can provide the resonance. Resonance may be unnecessary for operation of the non-self-resonant implant antenna, and instead the operation of this implant antenna may be based on impedance changes. In example embodiments, when the non-self-resonant implant antenna is used in a conductive medium comprising biological tissues then a current path may be generated between two electrodes of the implant antenna inside the conductive medium. The current path extends to the biological environment and generates a larger area with a current distribution in the biological medium. The size of the area is much larger than the physical size of the implant antenna. This give rise to a large virtual size for the implant antenna and thus high efficiency is achieved. In prior art using a self-resonant implant antenna, the implant antenna efficiency is limited to the physical size of the antenna geometry, whereas with a non-self-resonant implant antenna the implant antenna efficiency can be increased using the larger virtual size of the implant antenna.

The non-self-resonant implant antenna and the electrical switch(es) advantageously make use of the impedance of body tissue to affect the backscattering properties of the non-self-resonant implant antenna, with a two-part implant antenna having two electrodes arranged to be spaced apart and in contact with the body tissue when in use. The spacing between the electrodes may for example be from 5 mm to 35 mm.

The non-self-resonant implant antenna uses two conductive patches as electrodes that may form the main components of the non-self-resonant implant antenna. Metal patches may be used. The two electrodes can be arranged to be in contact with the body tissue, such as via direct tissue contact with the conductive material of the patches, or with an intervening layer of a thin bio-compatible non-conductive coating, such as a polymer coating. The layer of bio-compatible non-conductive material can allow for a gap in the coupling of the patch antennas to the body tissue, which can enhance operation of the implant antenna. The layer may be provided via a housing such as a shell placed around the antenna and other parts of the implant device. The housing or coating may encapsulate the implant device and serve to seal it to prevent the internal parts from direct exposure to body tissues and body fluids. This can also enhance the safety of the device and/or reduce the need for bio-compatible materials for the remainder of the device. The layer may for example have a thickness of 1 mm or less, such as a thickness of about 0.5 mm. In some examples a coating with a thickness of 0.1 mm or less may be used.

The backscattering properties of the non-self-resonant implant antenna are controlled by an electrical switch that is arranged to change the impedance of the non-self-resonant implant antenna by switching between coupling the two electrodes via the biological medium (e.g. body tissue) and coupling the two electrodes via a conductive pathway. Thus, in effect when the switch is open then a high impedance is provided due to coupling via the biological tissue, and when the switch is closed a low (nominally zero) impedance is provided via the conductive pathway through the switch. The large variation of the impedance can be identified by a receiving antenna placed on the body surface or under the skin. High efficiency of the reflections is achieved due to the large RCS, large bandwidth of the device and large impedance variations. This can generate a large reading range at depths up to 18 cm with a low/moderate transmitter power. High data rates of 12 Mbps have been measured and it is expected that a potential of 70 Mbps can be achieved.

In some examples the implant antenna uses two conductive patches providing two electrodes of the antenna and these patches are selectively coupled via a switch. The conductive patches may take various forms. They may be considered as patch antennas and hence as distinct from other antennas such as coil antennas or loop antennas.

The conductive patches may in one simple example be patches arranged for attachment to body tissue, whereby the attachment to the body tissue serves to fix the patches with a spacing between them. The switch may then connect or disconnect a conductive pathway between the patches so that the impedance of the body tissue has a lesser or greater effect on the effective impedance of the implant antenna.

The conductive patches may be spaced apart by virtue of their mounting to a part of the implant device, such as a body or a housing of the implant, with the two patches at different points along a length of the implant. In some examples the implant device has a tubular form, such as a cylindrical tube or a tube of any other prismatic shape, and the two conductive patches are mounted to the tube and spaced apart along the length of the tube. A tube is a convenient form for implant devices with various purposes, such as for capsule endoscopy.

One or both conductive patches may have a generally two dimensional form such as a rectangle or a disc. One or both conductive patches may have a three dimensional form such as a tube, a section of a sphere (e.g. a hemisphere) or some other three dimensional surface shape such as a surface of rotation of a curve.

In one example the implant device comprises two tube shaped conductive patches, which may advantageously be placed on a tube of the implant device, such as a tubular body of the implant. In another example the implant device comprises one tube shaped conductive patch and one patch with a differing shape, such as a disc or a non-tubular three dimensional form (e.g. a section of a sphere, for example a hemisphere). With this arrangement the tube conductive patch and the non-tube conductive patch may be placed on a tube of the implant device, for example with a disc on a flat end of the tube, or a hemisphere or other three dimensional form on a rounded end of the tube.

The conductive patches may take the form of tubes of conductive material, such as cylindrical tubes or rings. It is straightforward to manufacture and handle such tubes, and they can readily be inserted into body tissue in various ways. In addition, it is easy to place two conductive tubes (or rings) on a larger non-conductive tube in order to achieve the required distance between the tubes within the body as well as allowing for space to house the switching components and potentially also other elements of the implant device. The same approach can be used with two conductive patches of other shape mounted on a tube, as discussed above. In example embodiments the distance between the two conductive patches (e.g. the distance between two tubes) is limited to be 50 mm or below, optionally 35 mm or below. The distance between the conductive tubes may be at least 5 mm, and it may be in the range 5-35 mm as noted above. Cylindrical tubes such as rings may be used. The cylindrical tubes may have a diameter of at least 3 mm, optionally at least 4 mm, and in some examples a diameter in the range 3 mm to 15 mm. The conductive tubes may have a length along their axis of at least 1 mm, optionally at least 3 mm, and in some examples in the range 3 mm to 6 mm.

The implant device may use one or more switching devices for controlling the backscattering properties, and these may be low power electrical switching devices such as transistors. For example, Field-effect transistors (FET)s such as CMOS FETs may be used. In one example the transistor is a CMOS MMIC device that advantageously can operate with ultra low power. The use of a transistor provides a low power way to alter the backscatter properties of the implant antenna.

An internal power source of the implant device may be used to control the electrical switch(es) as well as also powering a data source. This internal power source may be a battery or optionally it may be a wireless power transfer system that can use the same implant antenna as the implant antenna used for backscatter communications. The implant device preferably does not take power from the implant antenna during the backscattered communication, which ensures that the implant antenna can be optimised for maximum reflection and hence maximum range for the backscatter communications with the on-body antenna apparatus. In fact a cycle of the data (0 or 1) can be used for power harvesting and the other cycle for data transmission.

There may be multiple electrical switches and filters having differing effects on the implant antenna's backscattering properties and hence allowing for more than two different states for the backscattered signal. This can allow for more complicated data transfer and higher data rates. The method becomes similar to frequency division multiplexing (FDM) for multiband data communication by shrinking high data rate in multiple frequencies. In addition, the switch and filter combination can separate the path for wireless power transfer and data transfer. For multiple frequency usage, the on-body antenna apparatus should transmit in the multiple frequencies of interest.

It will be understood that the above described system is a medical system for use with the human or animal body. The implant device may hence be a medical implant, such as a capsule endoscope or other medical implant. The system may use wireless transmission of data to transfer medical data from the implant to the on-body antenna, such as data relevant to the condition of the patient's body.

The implant device may include sensors for gathering data for medical use, such as use in medical diagnostic methods or for medical research. The sensors are preferably connected to a data source of the implant device in order to provide information used to generate data that may be transmitted from the data source to the on-body antenna apparatus. The implant device may additionally include additional data processing devices such as a computer processor and/or a memory for data storage.

As noted above it has been proposed in the prior art to combine backscatter communications with wireless power transfer from the on-body antenna apparatus to the implant device. The system may include a wireless power transfer capability. This reduces the range for backscatter within the body since the implant antenna must necessarily absorb some of and often a major part of the transmitted energy. With the present implant antenna the design of the implant antenna in the backscatter communication configuration may be for maximum reflection and hence minimal absorption of energy, and the implant device is advantageously arranged to use as much as possible of the energy incident on the antenna for backscatter communications. The implant device may be arranged such that there is no wireless power transfer during communication of data via the implant antenna. There may be a separate use of wireless power transfer when backscatter communication is not being carried out.

The data source or sensors of the implant device, where present, may have their own battery or a separate wireless power transfer system. The data source may be arranged to provide a data signal for controlling the backscattering properties of the implant antenna to thereby transmit data from the data source to the external communication system. Preferably this is a low power data signal thereby ensuring maximum lifetime for the implant device. By minimising the power requirement at the implant device the lifespan of the device is maximised whilst also allowing for a high data rate of the backscatter communications.

The invention further extends to a method including use of the system as in the first aspect, for example in order to gather medical information about a patient. The system may include any of the other features discussed above.

The use of the system may comprise using the on-body antenna apparatus to transmit an electromagnetic wave toward the implant device, which may have been implanted in the body in a prior procedure.

Optionally, the method may include using the implant device to gather medical information at one or more locations within the patient's body, and then transmitting this information out of the body via the wireless communications between the implant device and the on-body antenna apparatus. Where the implant device uses backscatter communications then the method may include controlling the backscattering properties of the implant antenna in order to thereby transmit data from a data source of the implant device using the backscattered signal; and receiving the backscattered signal at the on-body antenna apparatus. The use of the device may further include controlling the backscatter properties of the implant antenna to transmit data via the backscatter signal by means of switches as described above.

Certain preferred embodiments of the present invention will now be described by way of example only and with reference to the accompanying drawings, in which:

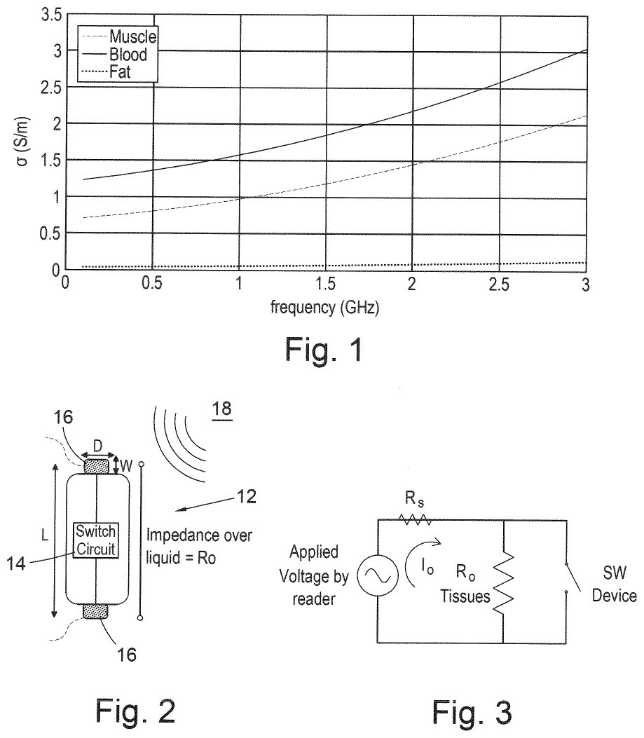

FIG. 1 is a plot of conductivity versus frequency for some biological tissues;

FIG. 2 shows an arrangement for galvanic electrodes as a non-self-resonant implant antenna;

FIG. 3 is a circuit model representing the operation of the switch in FIG. 2;

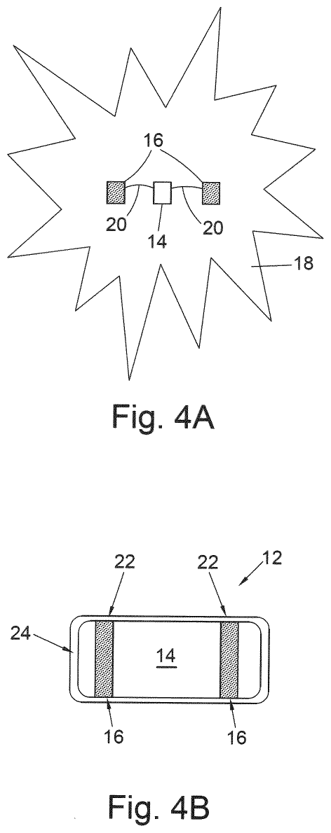

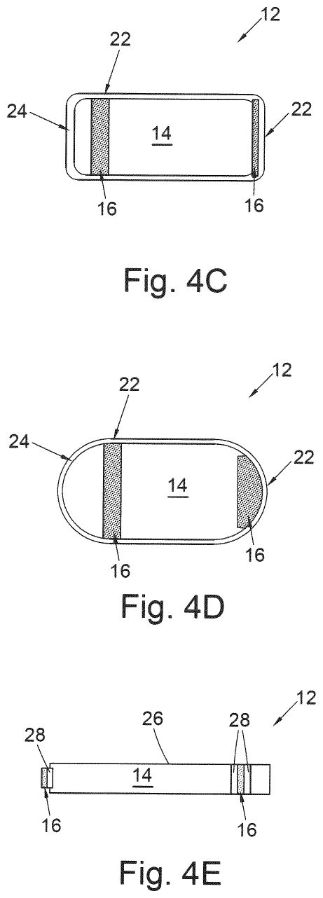

FIGS. 4A to 4E show various arrangements for the implant device and in particular for the conductive patches providing electrodes of the antenna;

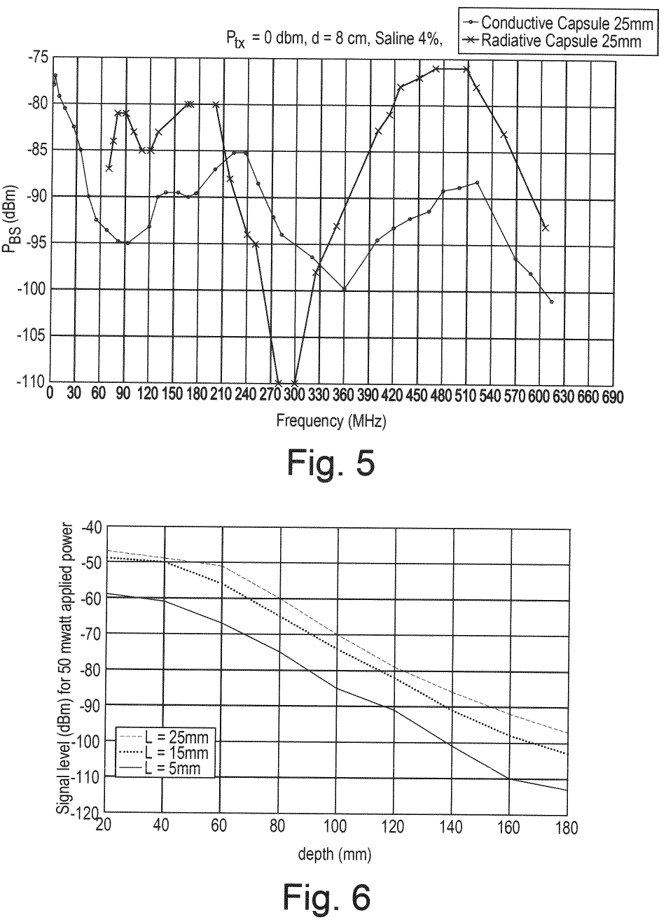

FIG. 5 shows test results with backscatter signal level plotted against frequency;

FIG. 6 shows the backscatter signal level versus depth for different capsule sizes 25, 15 and 5 mm at 455 MHz;

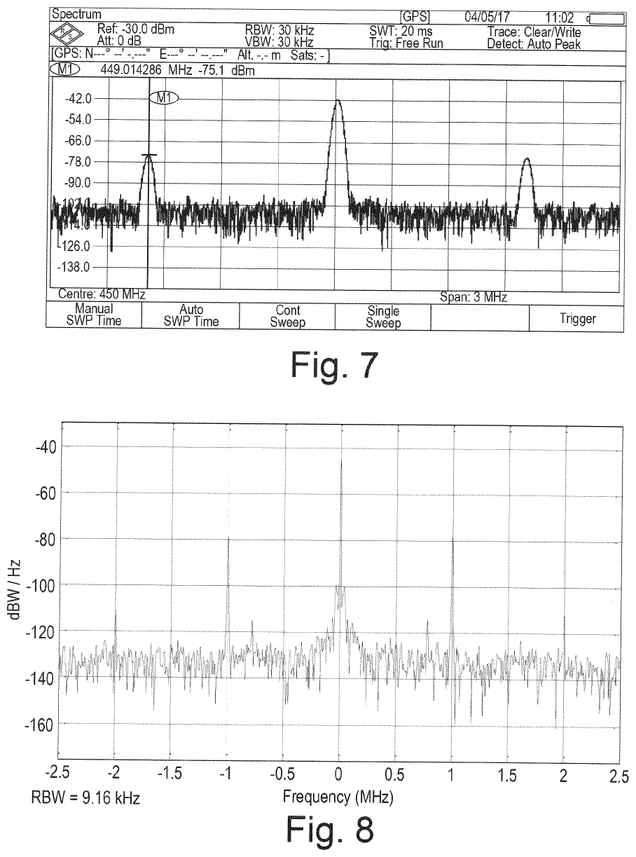

FIG. 7 shows a backscatter signal at a spectrum analyser for a test of a transmitted period signal with implant capsule at depth 9 cm;

FIG. 8 shows USRP spectrum analyser measurements for the same test;

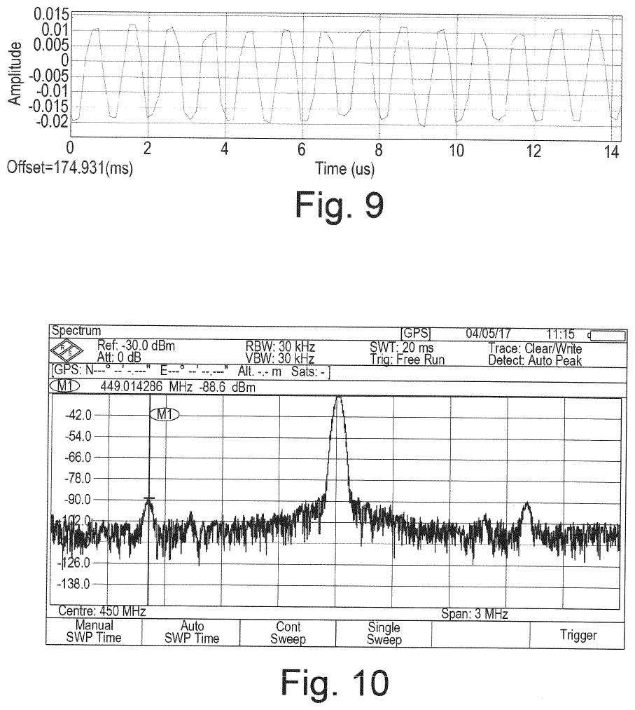

FIG. 9 shows the decoded data signal from the test of FIG. 8;

FIG. 10 shows the spectrum analyser measurements for a similar test with the implant capsule at a depth of 17 cm;

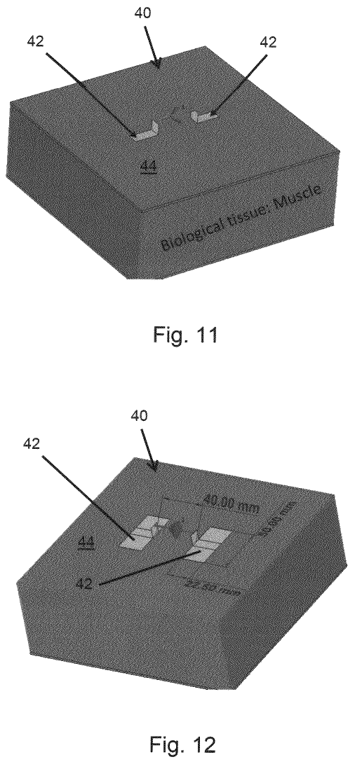

FIG. 11 shows a simulation model of an on-body antenna apparatus;

FIG. 12 shows a similar model to FIG. 11 for a different geometry of antenna patches;

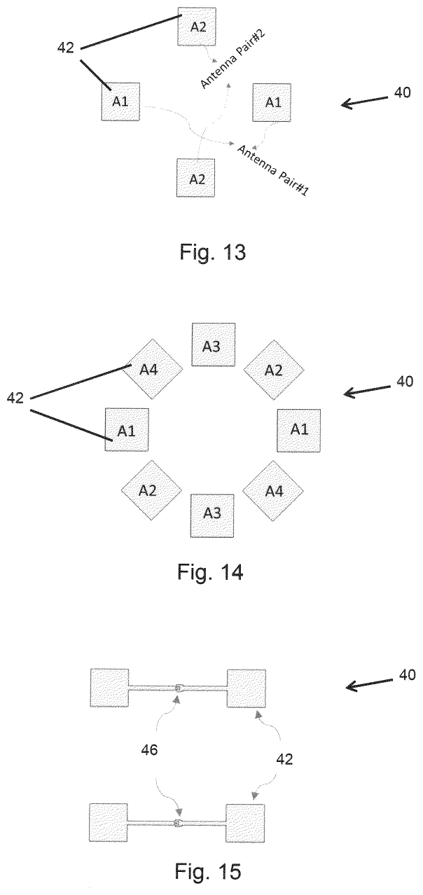

FIG. 13 illustrates an arrangement of patch antenna pairs for dually polarized wave coupling;

FIG. 14 shows multiple patch antenna pairs being used to give multiple different polarization angles;

FIG. 15 shows another arrangement for patch antenna pairs forming a space diversity antenna for the on-body antenna;

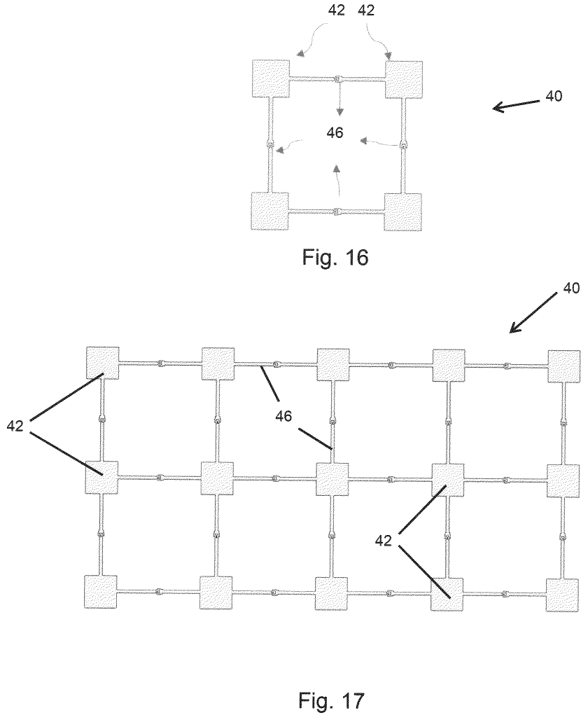

FIG. 16 is an arrangement similar to FIG. 15 with the addition of further antenna feed connections;

FIG. 17 illustrates a grid of electrodes to provide a large number of patch antenna pairs and enable extension of space and polarization diversity; and

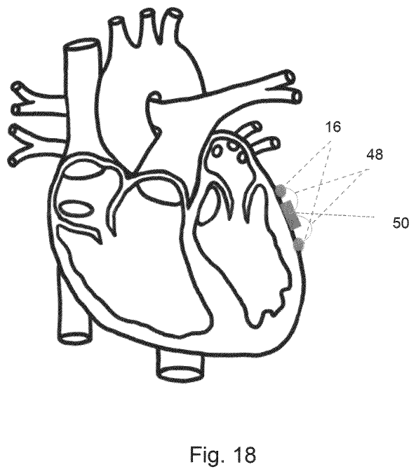

FIG. 18 shows a system in use for wireless monitoring using an implant device at the heart.

A system using an implant device with wireless transmission of power and/or data is described further below with reference to an on-body antenna apparatus that may be as shown in FIGS. 11 to 17. The implant device can be as discussed below such as with reference to the implant antenna arrangements of FIGS. 2 to 4E.

As noted above, the implant device may use a resonant antenna arrangement as disclosed in WO 2018/011235, or it may use a non-self-resonant implant antenna a described in more detail below. The implant antenna is arranged for backscatter communications. The proposed human body backscatter communication (HBBC) is appropriate for conductive mediums in which the regular backscatter communication and RFID techniques are hindered due to the tissues' loss and the implant antenna performance degradation. Similar to the backscatter techniques, our approach removes the transmitter from the implant device that can save the power consumption of the implant and physical space. A remote reader is used to transmit RF energy and read the information signal which is modulated by the implant's data source. Using this approach, the implant power consumption for communication reduces from several m-watt to some nano-watt thanks to using a switch circuit instead of a full transmitter chain in a conventional wireless communication. Thus high data rate and long-life connectivity are guaranteed with small implant devices.

The non-resonating antennas can provide ultra-high efficiency in the conductive mediums and ultra-wideband performance compared to the resonating antennas. Thus the link budget with the non-resonating antennas performs significantly better than the resonance base antennas. We implement the impedance of the media around the antenna for the antenna impedance tuning, and with the backscatter method the media's impedance is altered using the implant data signal and switch.

A reliable data connectivity with low to high rates (potential up to 70 Mbps) for the implant depths beyond 18 cm becomes feasible with a moderate transmitter power level (less than 100 m watts) of the remote reader. Therefore the system can be implemented for high depths connectivity. The implant size in our new approach can be reduced to 5 mm and it can be less (1 mm) for short distances. The method can be used for the implant to implant or implant to external body communication scenarios with potential applications in wireless cardiac pacemakers, wireless capsule endoscopy (WCE) and in-body wireless sensor network.

The regular wireless backscatter communication and RFID techniques are used in free space in which a remote reader transmits EM wave, and the received signal with a resonance antenna structure of a tag device is modulated and the signal is re-transmitted in the wave propagation channel. A switch circuit in the tag device performs the signal modulation. The remote reader demodulates the wave reflections for data detection. Operating at the tag resonance is essential to increase the antenna radar cross section (RCS) and provide appropriate backscatter link budget. The size of the tag antenna plays a crucial role on the system's performance, and by reducing the electrical size of the antenna from the resonance length (λ/4) the efficiency of the scheme is degraded.

Using the conventional backscatter and RFID methods are feasible for superficial medical implant communications for the sensing purpose. In this application, the communication range is small (1-3 cm), and the data rate is limited to some kbps. The main limitation is the implant size that should be low (<30 mm) and the frequency dependent loss of the biological tissues that increases with frequency. This imposes using the MHz frequency range (<1500 MHz). Therefore, the normalised antenna size becomes small (<λ/10) causing the antenna efficiency reduction. Furthermore, due to the small size, the antenna reactive field (near field) becomes significantly strong that is absorbed by the antenna surrounding tissues and give rise to the specific absorption rate (SAR) that reduces further the total antenna efficiency. These inherent limitations with the resonant antennas limit the application of RFID for biomedical implant communication.

Using antennas with large RCS and tuning the implant antenna resonance can help on increasing the communication range of the implant device. However, due to the small efficiency of an implanted antenna and the limitations on the applied reader's power (due to RF absorption and SAR) the communication range in earlier work with resonant antennas has been limited to about 8 cm with an applied power of 100 m watts. Also, the resonance frequency of the implant antenna shifts due to different tissues' loading that plays a crucial role in the backscatter performance. A complex system should be used to find the best frequency for the backscatter. Furthermore, the data rate that can be delivered with a small resonating antenna is limited.

The currently proposed approach is based on a backscatter mechanism which is adapted for the conductive or lossy mediums such as the biological environments. We use a non-self-resonant implant antenna with medium impedance modulation for realising human body backscatter communications (HBBC). The efficiency of the new antenna is not affected by the lossy material similar to the resonance antennas. The non-self-resonant implant antenna can provide appropriate impedance in the conductive mediums by exploiting the loss of the environment for matching. The antenna should have direct contact with the biological tissues, or it can hold a tiny gap (<0.5 mm) as coating with the surrounding material. As the antenna has a non self-resonance structure, the antenna efficiency is not a factor of the antenna electrical length. Thus the antenna can hold very small size with high efficiency. The near-field of the non-self-resonant implant antenna is not strong compared to the resonance structures thus the dissipated power around the antenna is significantly low. This introduces small specific absorption rate (SAR). All these features provide a reliable communication link with a remote reader for HBBC application that cannot be achieved with the conventional RFID and self-resonance techniques.

The implant media defines the impedance of the proposed non-self-resonant implant antenna instead of the antenna structure itself. As the medium impedance can be assumed constant for over 30% of centre frequency, we can define the antenna as frequency independent. The result is that the antenna becomes ultra-wideband and can perform from DC to several GHz frequency ranges. Consequently, the same antenna can be used for galvanic and radiative communications. Also, the potential data rate of the system becomes significantly high due to the available bandwidth with low distortion.

To implement the backscatter modulation using the non-resonating antenna, we alter the impedance between two loci of the implant electrodes in the medium through an internal switch mechanism. Thus, we can use the impedance of the environment and modulate it. Therefore, efficient backscatter performance is achieved. The backscatter device is ultra-wideband, and thus a wide range of frequencies (DC-1500 MHz) can be used for data communications in the biological tissues. Also, due to the available wideband feature, we can transmit very high data rate (70 Mbps at 450 MHz centre frequency). We note that the operation in all the frequency range is not similar and is not optimal. The operation mainly depends on the separation between the two electrodes of the antenna. Small separation imposes better operation at higher frequencies.

The proposed design is used for communications from implant to implant or from implant to an on-body antenna apparatus (as discussed below) with applications for wireless pacemakers and wireless capsule endoscopy (WCE). The disclosure herein provides the design and measurement results of the technology above. We describe the design concept of the new HBBC device, and the arrangement of the reader's antenna together with the system implementation. The measurements are conducted to provide the optimum frequency for HBBC, the effects of the device size and communication depths are measured.

The HBBC is presented for two possible scenarios:
Implant to implant communications for frequencies below 30 MHz with galvanic communications.
Implant to on-body communications based on the radiative coupling for higher frequencies such as those discussed below in relation to the on-body antenna apparatus.

Using backscatter for implant devices requires compact size implementation less than 35 mm. The frequency range that the biological tissues indicate small conductivity values (less loss) are below 1500 MHz (see FIG. 1 for a plot of conductivity versus frequency for some biological tissues). Therefore, the electrical size of an implanted antenna becomes less than λ/10 that is in the categories for the small antennas. The small antennas indicate strong near-field with high Q-factor and narrow bandwidth. Loading a small antenna with the lossy biological tissues such as the implant scenarios, dissipates most of the evanescent waves to the antenna surrounding tissues and thus reduces the antenna efficiency and the Q-factor drops. Therefore, using resonance antenna structure becomes problematic for the medical implant applications. This is the inherent character of any small antenna in the implant applications and indicates low efficiency.

FIGS. 2 and 3 show a design considered for biological conductive mediums, in particular for backscatter communications. The antenna 12 includes two metallic electrodes 16 with a small distance between the electrodes 16. A remote reader 18 receives signals from the antenna 12 via backscatter communications. The antenna 12 in free space shows an infinite impedance between the two electrodes 16. However, if the antenna immersed in a conductive medium, the device impedance is specified by the material characteristics between the two electrodes 16 and the separation of the electrodes 16. A gap between the electrodes 16 and the body tissue can also have an impact, such as the gap 22 provided by the optional shell 24 discussed below in relation to FIGS. 4B, 4C and 4D. Also, as the material for the biological tissues is frequency dependent the impedance between the electrodes 16 also becomes frequency dependent. The curves in FIG. 1 show that the conductivity can be considered as almost flat for about 30% of bandwidth at frequencies below 500 MHz. The proposed antenna 12 with two electrodes 16 can be used as an active transmitter or receiver for a broad range of frequencies (DC-1000 MHZ) with appropriate impedance matching. The antenna impedance for a capsule size larger than 5 mm is in a range that can be tuned effectively for wideband usage. The main problem with using the antenna as a direct transmitter is the resistance between the two electrodes 16, and this can be small depending on the tissues between the electrodes 16. The low resistance drains the transmitter's power and reduces the transmitter efficiency.

For the backscatter purpose, a wire connection between the antenna electrodes 16 with a switch circuit 14 between both electrodes 16 is used. By controlling the switch operation, the impedance of the medium between the two electrodes 16 can be modulated between zero ohms (for the switch on) and the medium's impedance (for switch off) defined by the conductivity (σ S/m) of the media and the separation between the electrodes 16. By reducing the conductivity (for example at fat tissues) the impedance between the electrodes 16 increases and with high conductive tissues (blood, muscle) the impedance reduces. Thus the antenna is easy to implement at lossy mediums in contrast to a resonating antenna. Therefore, if a remote reader provides a voltage difference in the medium between the two electrodes 16, the switching alters the current in the equivalent circuit that can be retrieved by the reader. Controlling the switch 14 with the implant data source expresses the signal modulation at the reader's side 18. As can be seen, the environment impedance switching is the basis for our backscatter communications and not the device self-impedance. Therefore, any signal and frequency that can provide an appropriate voltage difference between the two electrodes 16 can be used for the sensing purpose.

The electrodes 16 can be provided by cylindrical tubes (or rings) of conductive material which are shown in side view in FIG. 2, with the dimension W being a length/width of the tube along the axis of the cylinder and the dimension D being a diameter of the tubes. The dimension L shows the spacing between the two electrodes 16. We propose the distance between the electrodes 16 of range (5 mm<L<35 mm) for optimum link performance for all frequency range from DC-1000 MHz and the diameter of the electrodes 16 is proposed for (D>4 mm) and the strip width (tube length) of (W>1 mm). However, we can use smaller size L, D and W with some loss in the system performance.

FIGS. 4A to 4E show various arrangements for the implant device and in particular for the conductive patches providing electrodes of the antenna. These implant devices may be manufactured for HBBC measurement.

FIG. 4A shows the use of two conductive patches 16 for the antenna electrodes, with these patches being attached to body tissue 18. The switch circuit 14 is connected to the two patches 16 via wires 20. The attachment of the patches to the body tissue 18 is used to fix them at the required spacing.

The examples of FIGS. 4B to 4E differ from that of 4A in that a body of the implant device 12 is used to support the patches 16 and hence to provide spacing between the patches. The implant device 12 in these examples has a tubular form, which is shown in cross-section. The switch circuit 14 as well as the conductive pathway (20, not shown) is within the body of the implant device 12. The patches 16 forming electrodes of the antenna are placed at an outer part of the implant device 12.

In FIG. 4B the patches 16 take the form of two tubes at two ends of the implant device 12. In FIG. 4C the implant device 12 uses one tube shaped patch 16 and one disc-shaped patch 16. The disc-shaped patch 16 is at an end of the tubular form of the implant device. The example of FIG. 4D uses rounded ends for the implant device 12 and one of the patches 16 is a tube, whilst the other patch 16 is a curved surface fitted to one of the rounded ends. For example, this may be a section of a sphere. Optionally there is a gap 22 between the patches 16 (i.e. the electrodes) and the body tissue, with this gap 22 being provided by an outer shell 24. The gap 22 operates as a capacitive element. The use of a gap 22 in this way may be done for backscatter applications at high frequencies (e.g. above 70 MHz).

The implant device 12 might include a camera (not shown) or other sensors. With the use of a camera this could be placed at an end of the device where there is no patch 16, such as the left hand end of the implant device 12 of FIG. 4D.

For some applications it may be useful to have a conductive outer shell 26, as shown in FIG. 4E. In this case the two patches 16, which may be a disc-shaped patch 16 at one end and a tube-shaped patch 16 at the other end, should be shielded from the conductive outer shell 26 by insulating material 28. Relevant applications include those in which using the metal is required for reasons of biocompatibility and/or long life of the implant device 12. An example of this is for leadless cardiac pacemakers. The backscatter electrodes 16 should be isolated in RF (reader frequency) from the casing. The electrodes 16 can be in contact with the surrounded medium or with a coupling gap.

In the experiments to validate the proposed concept different implant capsules have been manufactured and measured inside body phantom filled with phantom material (for example saline 4%) for simulating the human body tissues. A water container of 50×30×30 cm³ was used as our test phantom and filled with the fluid. The on-body antennas were placed on the container's surface. The capsule antenna 12, by way of example using an antenna configuration as shown in FIG. 4B, was immersed in the liquid in front of the reader antennas and parallel with the reader's antenna surface. A wide range of view angles in front of the reader antennas can provide data connectivity with the implant capsule 12. To cover whole areas for the scenarios with unknown locations and orientations of the capsule device 12, multi-antenna usage is proposed with a selection mechanism for providing the connectivity. The measurements have been validated for the in-vivo animal experiments confirming similar performance as the liquid homogeneous phantom.

In the first measurement setup, we define the frequency range which is appropriate for HBBC application. We consider two different scenarios with a conductive and radiative link. In the radiative link, the on-body antenna is placed with a gap to the container's surface. The source power is 0 dBm (1 m watt) and the backscatter signal level is recorded for the capsule at depth=80 mm. A capsule size of 25 mm is used in the measurement process. FIG. 5 shows the backscatter signal level versus frequency. As shown, the frequency range of 420-550 MHz shows high backscatter signal level. Other frequencies at 90 and 180 MHz provides good signal level. For the frequencies at 300 MHz band the signal level drops. Here we are interested in the MICS and RFID frequency bands at 402 MHz and 450 MHz. We note that the transmitted signal is not modulated thus the regulations for the un-modulated signal is applied to our system. The received signal is modulated and has wideband property, but the signal level is less than −85 dBm. As the modulated signal level is too small, it can be accepted by the regulatory standards for transmission. For instance, UWB transmission with a signal level below −80 dBm is an unlicensed band. Therefore, there is not any bandwidth limitation with the reflected signals which are feeble.

In the second measurement for HBBC, the same scenario with the reader antennas inside the liquid phantom is measured. The reader antennas are in direct contact with the medium. As expected, the high-frequency components are attenuated due to the direct contact with the lossy medium. However, the low-frequency band (galvanic coupling) is dominant for the frequencies below 30 MHZ in which the backscatter signal is significant. Therefore, the galvanic part is useful for low frequencies. The available bandwidth is limited in the lower frequencies. The bandwidth would be enough for HBBC connectivity with low rate requirements about 30% of the transmitter frequency. The same set-up can be used for high data rate connectivity with transmitting at low frequency and receiving the backscatter signal at a higher frequency (upconverted reflections). For example, by transmitting in some kHz in the forward path, we can modulate the backscatter signal at a higher subcarrier rate (1-10 MHZ) in the backwards path to obtain larger bandwidth for the data. In this case, the reader in TX and RX has different frequencies. This method would be appropriate for wireless pacemaker usage. Also, we can consider galvanic coupling for the forward path and the radiation for the backwards path. By using this communication style, the transmitted signal is in kHz and the reflected signal is at high frequency (MICS frequency band). The requirement is that a fast switching at the backwards frequency must be operated.

Considering the frequency range of 420-550 MHz for the radiative HBBC, we have measured data connectivity with different capsule sizes. In this measurement scenario, SDR is used as the transmitter and receiver device. We applied a power level of 50 m watt to measure the connectivity within depth, and the backscatter signal quality is recorded. FIG. 6 shows the backscatter signal level versus depth for different capsule sizes 25, 15 and 5 mm at 455 MHz. The capsule of size 25 mm performs better than the smaller capsules because of the available higher induced voltage at the electrode ends and the larger differential RCS provided by the impedance switching. The capsule with 5 mm size performs about 15 dB worse than the 25 mm capsule. Increasing the capsule size for more than 25 mm will not increase the backscatter signal significantly. This finding indicates that we can reduce the capsule size to 5 mm or less depend on the applicable depth. The backscatter path loss at 455 MHz band is about 5 dB/cm for all capsules. The loss decay is smaller at close distances to the reader due to the available near field of the reader.

Using the applied power of 50 mwatt, we can provide a depth connectivity up to 18 cm with signal level of −95 dBm. The capsule with 5 mm size can operate to depths of around 13 cm. The data rate for these systems is around 12 Mbps. The system can provide high data rate up to 70 Mbps in a single channel with small distortion. Frequency division multiplexing (FDM) can be used for an improved receiver performance.

Operating at 650 MHz can provide an increased link performance by 10 dB for the small capsule of size 5 mm. Therefore we should make a tradeoff between frequency and size of the implant capsule. Small size capsules provides improved backscatter at high frequencies. The measurements have been conducted in in-vivo animal experiments using a pig under general anaesthesia. High data rate connectivity is illustrated for in-depth (12-18 cm) applications in the abdomen and chest of the animal for wireless capsule endoscopy and wireless pacemaker applications.

The measurement of backscatter with the galvanic capsule shows that the signal loss for one-way connectivity is maximum 1.5 dB/cm and is 2.5 dB/cm for the radiative links. The efficiency of the coupling between the on-body and capsule antenna for capsule size of 25 mm is −10 dB. The coupling with complete matching provides a link with 30 dB loss at 8 cm. This means by transmitting 100 m watt, the received power at the capsule is 100 microwatt. Considering an efficiency of 35% for a rectifiers circuit we can deliver 35 microwatt power to the implant at 400 MHz frequency band. We expect more power transfer for the conductive scenarios using the non-resonating antenna, as the loss is smaller in the lower frequencies by about 8 dB for 8 cm. Therefore the proposed non-self-resonant implant antenna 12 can be used for WPT.

In another example, measurements were taken with SDR at 450 MHz band. USRP N210 was used as the reader system, the TX frequency was set to 450 MHz and the power is 0 dBm. A capsule size (L) of 25 mm was used at 9 cm depth, and the capsule was switched at 1 Mbps. The backscatter signal at the spectrum analyser was measured as shown in FIG. 7. Similarly, the spectrum was measured on USRP spectrum analyser and this is shown in FIG. 8, and the data signal is decoded as shown in FIG. 9.

The measurements were also conducted for a depth of 17 cm with increasing TX power to 15 dBm (30 mwatt) and RX gain of 15 dB in which we can detect the data signal of 1 Mbps. Based on the USRP bandwidth of 20 MHz the same performance can be achieved for data rate up to 10 Mbps. The spectrum for depth 17 cm is shown in FIG. 10 with the BS level −88.6 dBm.

The relative rotation of the reader antenna and capsule antenna 12 can cause signal degradation due to the polarisation mismatch. For a relative polarisation angle of 45 degrees, the signal loss is 5 dB and is increased to 10 dB for 60 degrees of relative rotation. The signal further reduces for rotation angles above 60 degrees. Therefore, the arrangement of the reader antennas should be adjusted to compensate for the relative rotation by multi-antenna configuration.

An implantable device is proposed that is adapted for conductive mediums and that allows for effective backscatter communications with low power usage. The introduced antenna 12 is a non-self-resonant structure with two conductive electrodes 16 that makes use of the impedance of body tissue during the antenna operation. As the antenna 12 is a non-resonating device, the losses associated with the near field, in the small resonating antennas, is not a limiting factor. The non-self-resonant implant antenna 12 is frequency independent with very wide range of stable impedances with frequency. Therefore, ultra wideband performance with the antenna 12 can be obtained. For the backscatter operation, a switch mechanism 14 is used to alter the system impedance between the two electrodes 16. The modulation of the medium impedance is used to express the data and backscatter connectivity. The proposed system can offer very high data rate connectivity of 70 Mbps at 450 MHz centre frequency for depths beyond 18 cm. The system is tested with different capsule sizes of 5, 15, 25 mm and data connectivity up to 12 Mbps to depths 12-18 cm are demonstrated with 50 m watt of the applied power. Depending on the usage and application scenarios, the frequency ranges from DC to 1500 MHz can be used for the backscatter connectivity. The power consumption of the implant device reduces from m-watt to several nano-watts thanks to using a switchboard instead of a complete transmitter in the implant. The technology is appropriate for WCE and pacemaker or implant wireless sensors network connectivity.

The proposed technique can also provide analogue data connectivity for the implant device in case that a varactor diode is used as the switch instead of the switch circuit 14. The reason is that we use the medium impedance modulation and not the antenna impedance that can reduce the RCS of the antenna.

Therefore, by using the concept of data transfer with the medium impedance modulation, we can provide data connectivity for very deep implant devices with small applied power to the medium. One reason for this is that the system is not based on an antenna resonance in which the tissue loading diminishes the high Q value of a small antenna. Also, all the frequency range from DC to several GHz can be covered for the backscatter data transfer. Any method that can provide a voltage difference between the two electrodes 16 of the implant device can be used for the backscatter sensing.

Two frequency ranges are considered for generating the voltage difference between two loci in the conductive medium.

1—Low frequencies (below 30 MHz) in which the electric conduction is the main way of signal transmission in the medium.

2—High frequencies (frequencies above 80 MHz) based on radiation of the RF field into the conductive medium.

For electric conduction direct contact between the electrodes 16 and the conductive medium is essential. However, for radiative coupling, a gap between the reader 20 and the medium is required to induce the electric field in the environment. Also, the implant device in the radiative scenario can hold a spacer or a non-conductive coating. Different frequency bands that can provide high penetration inside the biological tissues and provide a larger voltage difference between the implant electrodes 16 are found through extensive measurements. A summary of the features and findings are as follows:
An implant device using a non-self-resonant implant antenna 12 with two electrodes 16 can operate for backscatter data connectivity for all frequency range from DC to GHz. The lower band is limited to several tens of kHz to prevent the ionic movements and biological cell damage. The upper band is limited by the material loss of the biological tissues nominally to below 1000 MHz.
The backscatter method is based on the medium impedance modulation and not the antenna impedance modulation used in the conventional RFID and free space backscatter.
There is no resonating antenna for the implant radiator. Consequently, the device size and Q-factor are not the criteria for backscatter in conductive mediums.
The media impedance modulation approach can be integrated into any shape of an implant device such as cylindrical or planar structures. The only requirement is to have enough separation between the electrodes 16 of the implant device to obtain a considerable impedance between electrodes 16.
The separation between the two electrodes 16 of a device defines the system impedance and performance. The separation depends on the available space of the implant. Lengths between 25-35 mm are optimal. However, it can be reduced to about 15 mm with some loss of performance. Small sizes are possible around 5 mm but operation at higher frequencies is recommended.
The thickness or diameter of the device is not a critical factor. The electrode surface can be in the order of mm² (exp. 2×2 mm²).
Two ways of energy transfer to the implant device are considered by using the reader: Galvanic coupling and radiative coupling.
The optimum frequency band for the galvanic conduction is proposed for the implant to implant communication for frequency range of DC to 30 MHz.
The galvanic HBBC can be used for the implant to an external device communication if the wave radiation around the body is not permitted.
The frequency range for radiative coupling is measured in the phantom and optimum frequency of operation is extracted: namely three frequency bands are distinguished: 90, 180 and 450 MHz. We can define the frequency range at 650 MHz for communication with small implants of size 5 mm. Similar results have been confirmed using in-vivo animal experiments.
For the galvanic method, a conductive contact between external electrodes 16 and the body is required. We can use a conductive gel to provide the connection. In the MHz range, the fatty tissues have less effect on the link performance loss.
As the implant system is frequency independent device, Ultra wide bandwidth is available for data transmission due to the nature of the backscatter phenomenon. The bandwidth is mainly limited by the backscatter signal amplitude dispersion. This problem can be easily solved by a matched filter. The available bandwidth is more than 70 MHz at the 450 MHz centre frequency for radiative coupling mechanism.
The transmitter is a single tone signal transmitter and follows the related regulations for the transmitter power. For example, at 450 MHz it can be up to 300 mwatts. The backscatter signal which is modulated is below −80 dBm and can be considered as UWB spectrum in the unlicensed band.
Using the galvanic method, the bandwidth is limited to about 20% without amplitude dispersion. It can be increased if a matched filter is used.
Using multi-frequency for multi-capsule implementation is possible by applying an RF filter in the switching path of the implant device.
Backscatter with a capsule of 25 mm and data rate of 12 Mbps is demonstrated for depth 8 cm and 1 m watt transmitted power. The power rating of 50 m watts can provide connectivity to the depth of 18 cm at the centre frequency of 450 MHz.
Similar connectivity and depth of communication can be achieved with the galvanic method. However, the data rate is limited due to the limited bandwidth.

As noted above, FIGS. 11-17 show features of possible on-body antenna arrangements that can be used in a system for wireless transmission of power and/or data between the on-body antenna apparatus 40 and the implant device antenna 16, which can be as discussed above.

The on-body antenna apparatus 40 is a physical element that is used to radiate electric signal oscillations which generate electromagnetic radiations. The EM radiations from a device should be matched to that of free space for propagation in which the antenna performs this task. The EM radiation can transfer energy from one device to another device in space. Antenna design plays a crucial role in an efficient power transfer from a source to space. The antenna design in free space is well understood and developed.

Suitable on-body antennas can be considered based on the understanding from antenna theory in free space. The difference with free space is that the biological mediums are conductive environments that impose significant loss to RF signals. As discussed above, biological material conductivity is a function of the tissue type and the operating frequency of the system. Tissues with high water contents and minerals show more conductivity than dry tissues and the conductivity increases with frequency. The conductivity of tissue material limits wave propagation in the biological mediums and adds the propagation loss. Due to the material loss, the wave propagation into the in-depth body is limited.

As discussed further below, an on-body antenna apparatus 40 is designed in a way that is specific for guiding EM wave and electric signals to an implant device within biological mediums. In this concept, the antenna self-structure resonance is not used for radiation. Instead, we use the human body and the conductivity of the biological medium to couple EM wave into the biological tissues and extend the wave propagation into the body rather than in free space. Using this approach we can couple the EM wave more efficiently into the body rather than into free space. The EM wave can penetrate more in-depth, can provide smaller SAR than the free-space antennas, handle wide impedance bandwidth, have smaller size and are easy for tuning into different frequency bands.

The proposed antenna geometry consists of patch antennas 42 in the form of at least two conductive electrodes 42 of any suitable geometrical shape: for example, rectangular, triangular or circular shapes fed by an electric source signal in a given frequency band. The two patches 42 are spaced apart and are placed with a gap of 0.5-5 mm above the biological surface 44. FIGS. 11 and 12 show sample models used for simulations. They each include an on-body antenna apparatus 40 with two patch electrodes 42 above a muscle tissue with a gap insulator. There should not be direct contact with the tissues, except if frequencies are below 20 MHz. For low frequencies, the signal transmission is based on the conductivity of the tissues. For frequencies above 20 MHz, a gap is required to couple the signal to the tissues.

The patch configuration couples EM wave using the gap capacitance to the biological medium. As the biological medium is conducive to RF signals, a current path is generated through the tissue medium between the two electrodes 42. The current path between the electrodes 42 constitutes a loop antenna configuration that radiates the EM wave into the biological tissues with minimum loss. However, in the free space region, theoretically, there is no current path in to radiate the EM wave in air. Therefore, a significant part of the applied power is directed to the tissues medium. The wave is thus further extended inside the conductive medium (biological tissue) rather than into free space.

The patch antenna input impedance is defined by the coupling gap and the complex impedance of the biological medium, which is mainly inductive. The impedance becomes real for a wide frequency range in which the capacitive and inductive reactances are eliminated. So the antenna input impedance becomes a real value. The real value depends on the frequency and the size of the electrodes, the distance between the electrodes and the biological tissues under the patches. Using these parameters, the input resistance can be tuned.

The amount of the resistance in the frequency range of 450-1000 MHz varies between 40-70 ohms for a patch distance of 40 mm and size of 10*10 mm². The reactive antenna impedance can be eliminated with a gap or patch size or shape tuning. For instance, with a gap of 1-2 mm between the patch electrodes 42 and the body surface 44, a distance of 40 mm and patch electrode 42 surface area of 20 by 10 mm, an antenna apparatus 40 with 50 ohms resistance and wideband matching in the range of 600-900 MHz is achieved. In this case, the antenna SAR (10 g) is 6 W/kg for 1 Watt applied power for all the frequencies in the range. The SAR reduces by increasing the patch size. However, the antenna field of view becomes wider. Using smaller patches, the field of view is narrow but larger local SAR is achieved.

The antenna impedance bandwidth is 20% of the centre frequency with Ra variation of about 10%. With the antenna size above, the antenna operating frequency can be tuned to the frequency band at 250-350 MHz by increasing the lateral size of the electrodes 42, as shown in FIG. 12, where it will be seen that the width of the patch electrode 42 is increased compared to FIG. 11. Therefore, the antenna resonance can be adjusted by the distance between the electrodes 42 and the patch size.

The wave transmission loss depends on the frequency. It is about 1.5-3 dB/cm in the muscle tissue for the frequency range of 100-800 MHz. The wave coupling efficiency or antenna efficiency for the in body coupled EM wave is −2 dB using the electrode antenna. So, only 35% of the applied power is radiated into the free space.

The proposed on-body antenna 40 using two patch electrodes 42 with a coupling gap is a single radiator with a polarization in the direction of the line connecting the two patch electrodes 42. By adding a second electrode pair (antenna) and by rotating the antenna by 90 degrees compared to the first antenna, the biological medium can be exposed to RF radiations with different polarization angle as shown in FIG. 13. This can be useful for the applications in that are polarization dependent, such as a wireless communication link in which the implant antenna orientation affects the link performance. In addition, as there is a separation between the electrodes 42 and each antenna has own defined SAR value, then using multiple pairs of patch electrodes 42 can distribute the local SAR value and a larger total power can be applied using multiple electrodes 42 for any application such as WPT, microwave hyperthermia, microwave imaging and so forth. Using multiple antennas is also applicable for co-phase summation of EM wave in depth inside the body by providing different signal phase values. Thus the EM wave can be focused in a specified location.

In another variation the number of the patch electrodes 42 can be increased by circularly rotating the patches. FIG. 14 shows an example of such an arrangement. In this example four pairs of electrodes 42 are used to apply the power with different polarization angles. An advantage of this is that larger total power can be applied for a specific SAR value. Thus the biological tissue is radiated with larger power for any required application, example: WPT, imaging, communications, etc.

In another feature, the pairs of patch electrodes 42 can be separated by space diversity as shown in FIG. 15. The two pairs of patch antennas include separate feed connections 46. However, the antenna near field is limited to the geometrical view angle of each electrode 42. An antenna separation about λ/10 or less can be applied to radiate a similar region with each patch antenna pair. In this implementation phase tuning between the antenna pairs 42, 42 can be used to manage/engineer the near-field exposure.

The space diversity antenna can be fed using a dual polarization scheme. FIG. 16 shows a feeding mechanism of two patch antenna pairs with four possible feed connections 46. In this implementation, the two parallel patch electrodes 42 can be fed simultaneously. Therefore, it is possible to have space diversity, and by switching between the various possible pairs of electrodes 42, then it is possible to add polarization switched diversity. This permits enhanced performance within a small space. One application of this might be for data communication or WPT in which the target implant antenna rotation can be random, and the switching mechanism can adjust the field polarization.

The space and polarization diversity can be extended by the use of a grid of patch electrodes 42 and multiple feed connections 46 as shown in FIG. 17. In this implementation, any pairs or multiple pairs can be used for communications. Such an arrangement allows for selection of space and polarization diversity using a large number of possible antenna pairs. An appropriate antenna control system can be used to switch between pairs in order to “search” for the antenna pair with the best

The on-body antenna apparatus 40 using a grid can use a square grid giving an array as in FIG. 17. This could be formed with a planar geometry and can be used on the body surface 44 by manufacturing on a flexible substrate material. The on-body antenna apparatus 40 can, for example, be placed on a belt type holder and fastened to the body surface during use.

The designed array antenna is used for communication and backscatter communication. With the backscatter, one pair of the electrodes is used as a transmitter and the implant data can be retrieved in the backscatter link using another electrode pairs, in the same or orthogonal polarization.

For each of the various proposed on-body antenna configurations discussed above the coupling value between the on-body antenna apparatus 40 and an implant antenna (e.g. via the implant electrodes 16) using a similar concept of the gap coupling mechanism is relatively high. Thus, the on-body antenna apparatus 40 can be used effectively for WPT to a medical implant device, as well as for receiving transmission of data from the implant device.

The implant electrode size should be small compared to the on-body antenna patch electrodes in order to obtain best results. For example, it is possible to obtain a power coupling of better than −25 dB for an implant capsule of length 35 mm at 7 cm depth. This permits very efficient power transfer to a medical implant, in which considering the RF to DC conversion efficiency (30%) can reach to −30 dB of efficiency. Considering, 2 Watt of the applied power (which is in the SAR norm level) the delivered power becomes +3 dBm (2 m watt). This amount of power is further than enough for an implant operation. The WPT can be conducted at 455 MHz frequency, in which the SAR is smaller than higher frequencies. For instance, SAR at 455 MHz is 5 W/kg for applied 1 Watt of the power. So considering a limitation of 20 W/kg, we can apply 4 watts on the body surface. Also, multi-frequency WPT is an efficient way to increase the RF-DC conversion efficiency, so the same antenna on the body surface can be applied to transmit several multi-carriers for WPT.

In one example, with reference to FIG. 18 we consider using the electrode antenna mechanisms to power up a sensory system in a heart implant. In this example the WPT includes two sections for powering and backscatter communication:

1—The implanted antenna system 16.

2—The external reader and powering device, which may be as discussed above in relation to FIGS. 11-17.

The implant antenna system conducts two tasks.

1—In WPT mode, the implant antenna 16 is used as the receiver for RF/LF signal reception and an electronic rectifier diode circuit is used to rectify the signal and provide DC power for running the sensory system.

2—In the communication mode, the implant antenna 16 is used by a suitable switching mechanism for the backscatter data communications, such as that described above.

The on-body antenna apparatus 40 is used in a wireless power mode to transmit a signal to the implant device via a specific on-body antenna configuration on the chest. An appropriate configuration is required to handle the power to the implant device. In the communication mode, the reader device transmits an RF signal and receives the implant data via backscatter communications.

The on-body antenna apparatus 40 for monitoring of the implant device at the heart may have two/four electrodes 42 attached to the surface of the chest with a short gap to the skin. It may transmit maximum 1 W power to power the implant device. The distance between the on-body antenna electrodes 42 and the angle of the on-body antenna electrodes 42 are used for maximum power transfer. The reader system may be battery powered and can handle power to the implant during the sensing process. Feedback from the implant can be used to adjust the applied power for minimum RF radiations and confirmation for WPT link operation.

Wires 48 couple the implant antenna electrodes 16 to a sensor device 50. The sensor device 50 includes sensory electronics arranged to use the received power of 1-5 mW to run a microcontroller and a sensor (e.g. an accelerometer) for a given period (for example, 1 min). The sensor data are stored in a memory at the implant device for offline reading, or it can be transmitted the same time as reading (real-time) using the backscatter switching mechanism. Advantageously, if power transfer and backscatter links are used at the same time (for the implant at the heart, or for any other system of the type described herein) then they may be separated via a frequency division system, in which one frequency is used for WPT and another frequency with low power link for data communications.

The implant antenna includes two or four patch electrodes 16 screwed to the heart surface. The sensory system of the sensor device 50 typically requires 2-3 volts of DC signal and consumes less than 100 micro Watt power. To handle the DC signal, rectifier diodes (voltage doubler/multiplier) are used along with a capacitor to be charged. The power is derived from the capacitor to run the electronics. If the power level is not sufficient, sequential sensing will be conducted to allow for a lower power requirement, otherwise continuous sensing is performed.

The example implant device in this case will use two frequencies, low frequency (<100 MHz) for WPT and RF for backscatter communications. A simple filter in the implant will be used to separate the power and data paths.

1. A system for wireless transmission of data and/or power using an on-body antenna apparatus and an implant device inside the body, the system comprising:
the implant device, which is for use within the body and comprises an implant antenna arranged to receive wirelessly transmitted power and/or to wirelessly transmit data;
the on-body antenna apparatus, which is arranged to transmit power and/or data acting as a radiative antenna, wherein the on-body antenna apparatus comprises a pair of patch antennas arranged to be placed on the surface of the body spaced apart from one another and to form an antenna circuit that is coupled by the body tissue around and between the patch antennas; and
an antenna control system for providing power to the on-body antenna apparatus and/or for handling communications between the on-body antenna apparatus and the implant antenna, wherein the antenna control system is arranged to drive the on-body antenna apparatus.

2. A system as claimed in claim 1, wherein the on-body antenna is arranged to couple RF energy to a conductive human body medium using a gap capacitance, and hence is arranged to be placed on the surface of the body with a gap between the patch antennas and the surface of the body.

3. A system as claimed in claim 1 or 2, wherein the gap between the body surface and the patch antennas is in the range 0.5 mm to 5 mm.

4. A system as claimed in claim 1, 2 or 3, wherein the on-body antenna apparatus is a broadband antenna that has an antenna bandwidth covering Medical Implant Communication Service (MICS) bands and also being appropriate for wideband/ultrawideband applications.

5. A system as claimed in any preceding claim, being arranged to operate with data and/or power transmission at frequencies in the range 50 MHz to 1400 MHz, optionally in the range 100 MHz to 800 MHz.

6. A system as claimed in claim 5, being configured for transmissions in a MICS band including frequencies in one or more bands known for medical implant use in the range 400 MHz-460 MHz.

7. A system as claimed in any preceding claim, wherein the maximum dimension of the patch antennas is less than 20% of the wavelength in free space at the intended operating frequency of the system.

8. A system as claimed in claim 5 or 6, wherein the on-body antenna patches have maximum dimensions in the range 5 mm to 100 mm, optionally 10 mm to 50 mm.

9. A system as claimed in any preceding claim, wherein the patch antennas are arranged to be placed with a spacing between the patch antennas in the range 5 mm to 60 mm, optionally 10 mm to 40 mm.

10. A system as claimed in any preceding claim, wherein the patch antennas include: a first flat conductive patch, such as a rectangular patch, for placement on the body surface; and an additional flat conductive patch extending away from the first flat conductive patch such that the patch antennas have an L-shape in cross section.

11. A system as claimed in any preceding claim, wherein the on-body antenna apparatus comprises multiple pairs of patches with different polarisation angles.

12. A system as claimed in any preceding claim, wherein the on-body antenna apparatus comprises a multiple pairs of patches forming an array.

13. A system as claimed in claim 12, wherein the array comprises ten or more pairs of patches, with each pair being spaced apart from adjacent pairs; and wherein the antenna control system is arranged to select one pair of patches within the array of pairs to use in relation to data or power communications with the implant device.

14. A system as claimed in claim 13, wherein the antenna control system is arranged to conduct a searching operation by switching between the pairs to find the pair of patch antennas giving the best connection to the implant device.

15. A system as claimed in any of claims 11 to 14, wherein the antenna control system is arranged to feed respective pairs of patch antennas using a dual polarization scheme.

16. A system as claimed in any preceding claim, wherein the implant device is capable of wireless transmission of data, the implant device includes a data source for providing the data to be transmitted to the on-body antenna, and the wireless transmission of data by the implant is done using a backscatter communication technique.

17. A system as claimed in any claim 16, wherein the implant device is arranged to control the backscattering properties of the implant antenna in order to thereby transmit data to the on-body antenna apparatus; and the backscattering properties of the implant antenna are controlled by one or more electrical switch(es) that change the load and/or geometry of the implant antenna.

18. A system as claimed in claim 17, comprising a non-self-resonant implant antenna for backscatter communications with the on-body antenna apparatus, the non-self-resonant implant antenna having at least two electrodes including two conductive patches that are arranged to be spaced apart when the implant device is in use;

19. A system as claimed in claim 18, wherein the implant device is arranged to control the backscattering properties of the non-self-resonant implant antenna in order to thereby transmit data from the data source to the external communications system; and wherein the implant device is arranged such that the backscattering properties of the non-self-resonant implant antenna are controlled by one or more electrical switch(es) including an electrical switch that is arranged to change the impedance of the non-self-resonant implant antenna by switching between coupling the at least two electrodes via body tissue and coupling the at least two electrodes via a conductive pathway.

20. A system as claimed in any preceding claim, wherein the implant device is a medical implant device and includes one or more sensor(s) for gathering data for medical use.

21. A system as claimed in claim 20, wherein the one or more sensor(s) are connected to a data source of the implant device in order to provide information used to generate data that is transmitted from the data source to the on-body antenna system.

22. A system as claimed in any preceding claim, wherein the implant device is a capsule endoscope.

23. A method including use of the system of any preceding claims for wireless transmission of data and/or power between the on-body antenna apparatus and the implant device.

24. A method as claimed in claim 23, comprising using the implant device to gather medical information at one or more locations within the patient's body, and then transmitting this information out of the body via the wireless communications between the implant device and the on-body antenna apparatus.
